(19) United States
(12) Patent Application Publication
(10) Pub. No.: US 2023/0183079 A1
(43) Pub. Date: Jun. 15, 2023

(54) METAL-ZEOLITE COMPOSITIONS PREPARED BY MECHANOCHEMICAL SYNTHESIS, AND METHODS OF USE

(71) Applicant: UT-Battelle, LLC, Oak Ridge, TN (US)

(72) Inventors: Zhenglong Li, Knoxville, TN (US); Meijun Li, Knoxville, TN (US)

(21) Appl. No.: 18/078,195

(22) Filed: Dec. 9, 2022

Related U.S. Application Data
(60) Provisional application No. 63/288,084, filed on Dec. 10, 2021.

(51) Int. Cl. C01B 39/38; C01B 39/06; C07C 1/20

(52) U.S. Cl. CPC C01B 39/38; C01B 39/06; C07C 1/20; C07C 2529/40

(57) ABSTRACT

A metal-zeolite composition comprising: (i) a zeolite phase; and (ii) a metal, other than aluminum or silicon, nanoscopically dispersed throughout said zeolite phase, wherein, if agglomerations of said metal are present, the agglomerations have a size of no more than 1 micron, wherein the zeolite may be, for example, a dealuminated zeolite, and the metal may be selected from transition metals, main group metals, and lanthanide metals. Also described herein is a method for producing the metal-zeolite composition in which a zeolite phase and metal salt are mixed and ground by a solvent-less ball milling process to produce an initial mixture, and calcining the initial mixture to produce the metal-zeolite composition. Further described herein is a method for converting an oxygen-containing organic species to a hydrocarbon, the method comprising contacting the species with the above described metal-loaded zeolite catalyst at a temperature of at least 100° C. and up to 550° C.

### CROSS REFERENCE TO RELATED APPLICATION

This application claims the benefit of priority from U.S. Provisional Application 63/288,084, filed on Dec. 10, 2021.

### GOVERNMENT SUPPORT

This invention was made with government support under Prime Contract No. DE-AC05-000R22725 awarded by the U.S. Department of Energy. The government has certain rights in the invention.

### FIELD OF THE INVENTION

The present invention generally relates to the production of metal-zeolite compositions and their use as catalysts, such as for the catalytic conversion of alcohols or aldehydes to hydrocarbons, and more particularly, to mechanochemical and solvent-less methods for preparing metal-zeolite compositions.

### BACKGROUND OF THE INVENTION

The present conventional approaches (e.g., wet impregnation or hand grinding) for producing metal-zeolite compositions are substantially limited in several ways. In particular, current approaches are generally incapable of producing industrial scale batches of metal-zeolite compositions with good reproducibility (low variability) between batches, particularly in terms of the level of integration of the metal in the zeolite. The conventional methods generally result in a higher than desirable level of agglomeration or heterogeneity of the metal and zeolite components, particularly for larger batches. Conventional methods also often rely on the use of solvents, which present an environmental hazard and raise costs. As indicated above, conventional methods are also generally incapable of producing metal-zeolite compositions in which the metal is highly dispersed on a microscopic or nanoscopic level throughout the zeolite phase. There would be a significant benefit in the zeolite industry for a method that could produce more homogeneously dispersed metal-zeolite product on a large scale and in reproducible fashion and without the use of solvents.

### SUMMARY OF THE INVENTION

The present disclosure is foremost directed to a novel method for producing a metal-zeolite composition. The method is advantageously capable of producing large scale (i.e., industrial scale) batches of metal-zeolite compositions in a highly reproducible manner with the metal highly dispersed throughout the zeolite phase on a nanoscopic level. The method can also advantageously be practiced without the use of a solvent. In specific embodiments, the method includes the following steps: (i) mixing and grinding a zeolite phase and a metal salt by a solvent-less ball milling process to produce an initial mixture; and (ii) calcining the initial mixture to produce the metal-zeolite composition, wherein the metal in the metal-zeolite composition is nanoscopically dispersed throughout the zeolite phase, wherein, if agglomerations of the metal are present, the agglomerations have a size of no more than 1 micron. In some embodiments, the agglomerations have a size of no more than 0.5, 0.2, or 0.1 micron, or the metal is atomically dispersed throughout the zeolite phase (i.e., with no agglomerations). In particular embodiments, the method is based on a ball milling process, wherein the ball milling process may employ a speed of at least 150, 200, 250, 300, 350, or 400 rpm for at least 5, 10, 15, 20, 25, 30, 45, 60, or 90 minutes, or a speed and/or time within ranges bounded by any two of the foregoing values. In some embodiments, the ball milling process employs at least two different ball sizes, typically where one ball size is one-one-half, one-third, or one-quarter the size of another ball size, such as about 5 mm and about 15 mm (or about 4 mm and 12 mm, or about 6 mm and 18 mm). In some embodiments, the zeolite phase is a low-aluminum or dealuminated zeolite phase. In some embodiments, the zeolite phase may be ZSM-5 or a mesoporous zeolite such as MCM-41, any of which may or may not be low-aluminum or dealuminated.

In some embodiments, the metal-zeolite composition, described above, is used as a catalyst for the conversion of an oxygen-containing organic species (e.g., alcohol or aldehyde) into a hydrocarbon fraction. The method may include the step of contacting the oxygen-containing organic species with the metal-containing zeolite catalyst at a temperature of at least 100° C. and up to 550° C. to result in the oxygen-containing organic species being converted to the hydrocarbon fraction. In some embodiments, by virtue of the nanoscopic homogeneous dispersion of the metal throughout the metal-containing zeolite, the metal-containing zeolite provides a higher conversion and/or selectivity than can be achieved with a conventionally produced metal-zeolite composition, with all conditions otherwise the same.

### BRIEF DESCRIPTION OF THE DRAWINGS

FIG. 1. Ethanol upgrading reaction benchmarked for Cu—Y-Beta catalysts synthesized via mechanochemical process vs. hand-grinding method in different scale. 1Cu-7Y-Beta-GH-1 and 1Cu-7Y-Beta-GH-2 were prepared via hand-grinding method using 1 g and 2 g, respectively; 1Cu-7Y-Beta-BM-3 was prepared via mechanochemical process at 3 g scale.

FIG. 2 X-ray diffraction patterns for catalysts that were synthesized via mechanochemical process.

### DETAILED DESCRIPTION OF THE INVENTION

The metal-zeolite composition described herein includes a zeolite portion (i.e., zeolite phase) and a metal loaded into the zeolite. The zeolite considered herein can be any of the porous aluminosilicate structures (or dealuminated versions thereof) known in the art that are stable under high temperature conditions, i.e., of at least 100° C., 150° C., 200° C., 250° C., 300° C., and higher temperatures up to, for example, 500° C., 550° C., 600° C., 650° C., 700° C., 750° C., 800° C., 850° C., or 900° C. In particular embodiments, the zeolite is stable from at least 100° C. and up to 700° C. Typically, the zeolite is ordered by having a crystalline or partly crystalline structure. The zeolite can generally be described as a three-dimensional framework containing silicate (SiO₂ or SiO₄) and aluminate (Al₂O₃ or AlO₄) units that are interconnected (i.e., crosslinked) by the sharing of oxygen atoms.

The zeolite can be microporous (i.e., pore size of less than 2 μm), mesoporous (i.e., pore size within 2-50 μm, or sub-range therein), or a combination thereof. In some embodiments, the zeolite phase is completely or substantially microporous. By being completely or substantially microporous, the pore volume due to micropores can be, for example, 100%, or at least 95%, 96%, 97%, 98%, 99%, or 99.5%, with the remaining pore volume being due to mesopores, or in some embodiments, macropores (pore size greater than 50 μm). In other embodiments, the zeolite phase is completely or substantially mesoporous. By being completely or substantially mesoporous, the pore volume due to mesopores can be, for example, 100%, or at least 95%, 96%, 97%, 98%, 99%, or 99.5%, with the remaining pore volume being due to micropores, or in some embodiments, macropores. In yet other embodiments, the zeolite phase contains an abundance of both micropores and mesopores. By containing an abundance of both micropores and mesopores, the pore volume due to mesopores can be, for example, up to, at least, or precisely 50%, 60%, 70%, 80%, or 90%, with the pore volume balance being due to micropores, or vice-versa.

The zeolite can have any suitable silica-to-alumina (i.e., SiO₂/Al₂O₃ or “Si/Al”) ratio. In some embodiments, the zeolite composition is partially dealuminated and has a silicon to aluminum ratio of at least or above 10, or the dealuminated zeolite composition does not contain aluminum (i.e., is completely dealuminated and composed of only silicon oxide, and optionally, one or more other elements in a trace amount). In various embodiments, the zeolite can have a Si/Al ratio of precisely, at least, more than, less than, or up to 10, 11, 12, 13, 14, 15, 20, 25, 30, 35, 40, 45, 50, 55, 60, 65, 70, 75, 80, 85, 90, 95, 100, 120, 150, 200, 300, 400, 500, 600, 700, 800, 900, 1000, 1100, 1200, 1300, 1400, or 1500, or a Si/Al ratio within a range bounded by any two of the foregoing values. As aluminum is present in any of the foregoing zeolite compositions having any of the recited Si/Al ratios, any such zeolite is herein considered to be partially dealuminated. The zeolite may also be completely dealuminated, in which case the zeolite does not contain aluminum, and thus, cannot have a Si/Al ratio. In some embodiments, the zeolite is at least partially dealuminated and has a Si/Al ratio of at least or above 10, 15, 20, 25, or 30, including any of the Si/Al ratios over 30 provided above.

In various embodiments, the zeolite (whether aluminated, partially dealuminated, or completely dealuminated) is a MFI-type zeolite, MWW-type zeolite, MEL-type zeolite, MTW-type zeolite, MCM-type zeolite, BEA-type zeolite, kaolin, or a faujasite-type of zeolite. Some particular examples of zeolites include the pentasil zeolites, and more particularly, the ZSM class of zeolites (e.g., ZSM-5, ZSM-8, ZSM-11, ZSM-12, ZSM-15, ZSM-23, ZSM-35, ZSM-38, ZSM-48), zeolite X, zeolite Y, zeolite beta, and the MCM class of zeolites (e.g., MCM-22, MCM-41, and MCM-49). The compositions, structures, and properties of these zeolites are well-known in the art, and have been described in detail, as found in, for example, U.S. Pat. Nos. 4,721,609, 4,596,704, 3,702,886, 7,459,413, and 4,427,789, the contents of which are incorporated herein by reference in their entirety. In particular embodiments, the zeolite is ZSM-5. ZSM-5 belongs to the pentasil-containing class of zeolites, all of which are also considered herein. In particular embodiments, the ZSM-5 zeolite is represented by the formula NaₙAlₙSi₉₆₋ₙO₁₉₂·16H₂O, wherein 0<n<27. In some embodiments, the zeolite is a 2D pillared zeolite, as well known in the art. The 2D pillared zeolite can be a 2D pillared version of any of the zeolites described above, such as a pillared MFI or MWW zeolite.

Typically, the zeolite contains an amount of cationic species, aside from active metal species. As is well known in the art, the amount of cationic species is generally proportional to the amount of aluminum in the zeolite. This is because the replacement of silicon atoms with lower valent aluminum atoms necessitates the presence of counter-cations to establish a charge balance. Some examples of cationic species include hydrogen ions (W), alkali metal ions, alkaline earth metal ions, and main group metal ions. Some examples of alkali metal ions that may be included in the zeolite include lithium (Li⁺), sodium (Na⁺), potassium (K⁺), rubidium (Rb⁺), and cesium (Cs⁺). Some examples of alkaline earth metal ions that may be included in the zeolite include (Be²⁺), magnesium (Mg²⁺), calcium (Ca²⁺), strontium (Sr²⁺), and barium (Ba²⁺). Some examples of main group metal ions that may be included in the zeolite include boron (B³⁺), gallium (Ga³⁺), indium (In³⁺), and arsenic (As³⁺). In some embodiments, a combination of cationic species is included. The cationic species can be in a trace amount (e.g., no more than 0.01 or 0.001%), or alternatively, in a significant amount (e.g., above 0.01%, and up to, for example, 0.1, 0.5, 1, 2, 3, 4, or 5% by weight of the zeolite). In some embodiments, any one or more of the above classes or specific examples of cationic species are excluded from the zeolite.

Generally, the zeolite described herein is in the form of a powder. In a first set of embodiments, at least a portion, or all, of the particles of the powder have a size less than a micron (i.e., nanosized particles). The nanosized particles can have a particle size of precisely, at least, up to, or less than, for example, 1, 2, 5, 10, 20, 30, 40, 50, 60, 70, 80, 90, 100, 150, 200, 250, 300, 350, 400, 450, 500, 550, 600, 650, 700, 750, 800, 850, 900, or 950 nanometers (nm), or a particle size within a range bounded by any two of the foregoing values. In a second set of embodiments, at least a portion, or all, of the particles of the powder have a size at or above 1 micron in size. The micron-sized particles can have a particle size of precisely, at least, up to, or less than, for example, 0.25, 0.5, 1, 2, 5, 10, 20, 30, 40, 50, 60, 70, 80, 90, or 100 microns (μm), or a particle size within a range bounded by any two of the foregoing values. In some embodiments, single crystals or grains of the zeolite correspond to any of the sizes provided above, while in other embodiments, crystals or grains of the zeolite are agglomerated to provide agglomerated crystallites or grains having any of the above exemplary dimensions.

In other embodiments, the zeolite can be in the form of a film, a coating, or a multiplicity of films or coatings. The thickness of the coatings or multiplicity of coatings can be, for example, 1, 2, 5, 10, 50, or 100 microns, or a range therein, or up to 100 micron thickness. In yet other embodiments, the zeolite is in the form of a non-particulate (i.e., continuous) bulk solid. In still other embodiments, the zeolite can be fibrous or in the form of a mesh.

The zeolite (with metal incorporated therein) can also be mixed with or affixed onto a support material, such as one suitable for operation in a catalytic converter. The support material can be a powder (e.g., having any of the above particle sizes), granular (e.g., 0.5 mm or greater particle size), a bulk material, such as a honeycomb monolith of the flow-through type, a plate or multi-plate structure, or corrugated metal sheets. If a honeycomb structure is used, the honeycomb structure can contain any suitable density of cells. For example, the honeycomb structure can have 100, 200, 300, 400, 500, 600, 700, 800, or 900 cells per square inch (cells/in²) (or from 62-140 cells/cm²) or greater. The support material is generally constructed of a refractory composition, such as those containing cordierite, mullite, alumina (e.g., α-, β-, or γ-alumina), zirconia, or a carbide (e.g., silicon carbide), or a combination thereof. Honeycomb structures, in particular, are described in detail in, for example, U.S. Pat. Nos. 5,314,665, 7,442,425, and 7,438,868, the contents of which are incorporated herein by reference in their entirety. When corrugated or other types of metal sheets are used, these can be layered on top of each other with catalyst material supported on the sheets such that passages remain that allow the flow of alcohol-containing fluid. The layered sheets can also be formed into a structure, such as a cylinder, by winding the sheets.

The zeolite described above is loaded with an amount of metal. Typically, the metal loaded into the zeolite is selected such that the resulting metal-loaded zeolite is catalytically active, such as for converting an oxygen-containing species (e.g., an alcohol or aldehyde) to a hydrocarbon fraction. Typically, the metal considered herein is in the form of positively-charged metal ions (i.e., metal cations). The metal cations can be, for example, monovalent, divalent, trivalent, tetravalent, pentavalent, or hexavalent. In some embodiments, the metal is (or includes) one or more types of alkali metal ions (e.g., Li, Na, and/or K). In other embodiments, the metal is (or includes) one or more types of alkaline earth metal ions (e.g., Be, Mg, Ca, and/or Sr). In other embodiments, the metal is (or includes) a transition metal, which may be any one or more elements of Groups 3-12 of the Periodic Table, and which may be a first, second, or third row transition metal. Some typical transition metals include copper, iron, zinc, titanium, vanadium, yttrium, zirconium, niobium, hafnium, and cadmium. The copper ions can be cuprous (Cu⁺¹) or cupric (Cu⁺²) in nature, and the iron atoms can be ferrous (Fe⁺²) or ferric (Fe⁺³) in nature. Vanadium ions may be in any of its known oxidation states, e.g., V⁺², V⁺³, V⁺⁴, and V⁺⁵. In other embodiments, the metal is (or includes) a main group metal (e.g., any one or more of elements of Groups 13-16 of the Periodic Table), such as gallium or indium. In other embodiments, the metal is (or includes) one or more types of lanthanide metals, which may be any one or more elements having an atomic number of 57-71 (e.g., La, Ce, Pr, Nd or other lanthanide). A single metal or a combination of metals may be loaded into the zeolite. In other embodiments, any one or more classes or specific types of metals described above are excluded from the zeolite.

The metal loading can be any suitable amount, wherein the loading is typically expressed as the amount of metal by weight of the zeolite. In different embodiments, the metal loading is precisely, at least, less than, or up to, for example, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 3%, 4%, 5%, 6%, 7%, 8%, 9%, 10%, 11%, 12%, 13%, 14%, 15%, 16%, 17%, 18%, 19%, or 20%, or a metal loading within a range bounded by any two of the foregoing values (e.g., 1-20%, 1-15%, 1-10%, 1-5%, 2-5%, 2-4%, 5-20%, 5-15%, or 5-10%). In some embodiments, a single metal is present in the zeolite, and the single metal may be in any of the amounts or ranges provided above. In other embodiments, more than one metal is present in the zeolite, and the metals may each independently be present in any of the amounts or ranges thereof provided above, or the total amount of metal corresponds to any of the amounts or ranges provided above. Any of the foregoing amounts or ranges thereof may correspond to an amount of one or a combination of alkali, alkaline earth, main group, transition metal, or lanthanide metals, or the amount or range may correspond to the total amount of metals in the zeolite composition (other than silicon and aluminum).

In other aspects of the invention, the zeolite composition may include at least one trivalent metal ion alone or in addition to one or more metals described above (except that Al may be substantially or completely absent). As used herein, the term “trivalent metal ion” is defined as a trivalent metal ion other than aluminum (Al⁺³). The trivalent metal ion may be a main group, transition metal, or lanthanide metal. Without wishing to be bound by any theory, it is believed that the trivalent metal is incorporated into the zeolite structure. More specifically, the incorporated trivalent metal ion is believed to be bound in the zeolite to an appropriate number of oxygen atoms, i.e., as a metal oxide unit containing the metal cation connected to the structure via oxygen bridges. In some embodiments, the presence of a trivalent metal ion in combination with one or more other catalytically active metal ions may cause a combined effect different than the cumulative effect of these ions when used alone. The effect primarily considered herein is on the resulting catalyst's ability to convert alcohols into hydrocarbons.

In some embodiments, only one type of trivalent metal ion aside from (or in the absence of) aluminum is incorporated into the zeolite. In other embodiments, at least two types of trivalent metal ions aside from aluminum are incorporated into the zeolite. In yet other embodiments, at least three types of trivalent metal ions aside from aluminum are incorporated into the zeolite. In yet other embodiments, precisely two or three types of trivalent metal ions aside from aluminum are incorporated into the zeolite.

In a first set of embodiments, at least one trivalent metal ion is selected from trivalent transition metal ions. The one or more transition metals can be selected from any or a select portion of the following types of transition metals: elements of Group 3 (Sc group), Group 4 (Ti group), Group 5 (V group), Group 6 (Cr group), Group 7 (Mn group), Group 8 (Fe group), Group 9 (Co group), Group 10 (Ni group), Group 11 (Cu group), or Group 12 (Zn group) of the Periodic Table of the Elements. Some examples of trivalent transition metal ions include Sc⁺³, Y⁺³, V⁺³, Nb⁺³, Cr⁺³, Fe⁺³, and Co⁺³. In other embodiments, the trivalent metal ion excludes all transition metal ions, or alternatively, excludes any one, two, or more classes or specific examples of transition metal ions provided above. In particular embodiments, the trivalent transition metal ions include Sc⁺³, Y⁺³ or Fe⁺³, or a combination thereof.

In a second set of embodiments, at least one trivalent metal ion is selected from trivalent main group metal ions. The one or more main group metals can be selected from any or a select portion of elements of Group 13 (B group) and/or Group 15 (N group) of the Periodic Table, other than aluminum. Some examples of trivalent main group metal ions include Ga⁺³, In⁺³, As⁺³, Sb⁺³, and Bi⁺³. In other embodiments, the trivalent metal ion excludes all main group metal ions other than aluminum, or alternatively, excludes any one, two, or more classes or specific examples of main group metal ions provided above. In particular embodiments, the trivalent main group metal ions include at least In³⁺.

In a third set of embodiments, at least one trivalent metal ion is selected from trivalent lanthanide metal ions. Some examples of trivalent lanthanide metal ions considered herein include La⁺³, Ce⁺³, Pr⁺³, Nd⁺³, Sm⁺³, Eu⁺³, Gd⁺³, Tb⁺³, Dy⁺³, Ho⁺³, Er⁺³, Tm⁺³, Yb⁺³ and Lu⁺³. In particular embodiments, the trivalent lanthanide metal ion is selected from one or a combination of La⁺³, Ce⁺³, Pr⁺³, and Nd⁺³. In further particular embodiments, the trivalent lanthanide metal ion is or includes La⁺³. In other embodiments, the trivalent metal ion excludes all lanthanide metal ions, or alternatively, excludes any one, two, or more classes or specific examples of lanthanide metal ions provided above.

In a fourth set of embodiments, the zeolite includes at least two trivalent metal ions selected from trivalent transition metal ions. Some combinations of trivalent transition metal ions considered herein include Sc⁺³ in combination with one or more other trivalent transition metal ions, or Fe⁺³ in combination with one or more other trivalent transition metal ions, or Y⁺³ in combination with one or more other trivalent transition metal ions, or V⁺³ in combination with one or more other trivalent transition metal ions.

In a fifth set of embodiments, the zeolite includes at least two trivalent metal ions selected from trivalent main group metal ions. Some combinations of trivalent main group metal ions considered herein include In⁺³ in combination with one or more other trivalent main group metal ions, or Ga⁺³ in combination with one or more other trivalent main group metal ions, or As⁺³ in combination with one or more other trivalent main group metal ions.

In a sixth set of embodiments, the zeolite includes at least two trivalent metal ions selected from trivalent lanthanide metal ions. Some combinations of trivalent lanthanide metal ions considered herein include La⁺³ in combination with one or more other trivalent lanthanide metal ions, or Ce⁺³ in combination with one or more other trivalent lanthanide metal ions, or Pr⁺³ in combination with one or more other trivalent lanthanide metal ions, or Nd⁺³ in combination with one or more other trivalent lanthanide metal ions.

In a seventh set of embodiments, the zeolite includes at least one trivalent transition metal ion and at least one trivalent lanthanide metal ion. For example, in particular embodiments, at least one trivalent metal ion is selected from Sc⁺³, Fe⁺³, V⁺³, and/or Y⁺³, and another trivalent metal ion is selected from La⁺³, Ce⁺³, Pr⁺³, and/or Nd⁺³.

In an eighth set of embodiments, the zeolite includes at least one trivalent transition metal ion and at least one trivalent main group metal ion. For example, in particular embodiments, at least one trivalent metal ion is selected from Sc⁺³, Fe⁺³, V⁺³, and/or Y⁺³, and another trivalent metal ion is selected from In⁺³, Ga⁺³, and/or In⁺³.

In a ninth set of embodiments, the zeolite includes at least one trivalent main group metal ion and at least one trivalent lanthanide metal ion. For example, in particular embodiments, at least one trivalent metal ion is selected from In⁺³, Ga⁺³, and/or In⁺³, and another trivalent metal ion is selected from La⁺³, Ce⁺³, Pr⁺³, and/or Nd⁺³.

In a tenth set of embodiments, the zeolite includes at least three trivalent metal ions. The at least three trivalent metal ions can be selected from trivalent transition metal ions, trivalent main group metal ions, and/or trivalent lanthanide metal ions.

In particular embodiments, one, two, three, or more trivalent metal ions are selected from Sc⁺³, Fe⁺³, V⁺³, Y⁺³, La⁺³, Ce⁺³, Pr⁺³, Nd⁺³, In⁺³, and/or Ga⁺³. In more particular embodiments, one, two, three, or more trivalent metal ions are selected from Sc⁺³, Fe⁺³, V⁺³, Y⁺³, La⁺³, and/or In⁺³.

Any of the above trivalent metal ions can be included in the zeolite composition in any suitable amount, such as, precisely, at least, less than, or up to, for example, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 3%, 4%, 5%, 6%, 7%, 8%, 9%, 10%, 11%, 12%, 13%, 14%, 15%, 16%, 17%, 18%, 19%, or 20%, or a metal loading within a range bounded by any two of the foregoing values (e.g., 1-20%, 1-15%, 1-10%, 1-5%, 2-5%, 2-4%, 5-20%, 5-15%, or 5-10%). Alternatively, the total amount of trivalent metal ions (other than Al) may be limited to any of the foregoing values. In some embodiments, one or more specific types, or all, trivalent metal ions other than Al are excluded from the catalyst.

In particular embodiments, the zeolite is or includes a pentasil-type composition loaded with any of the suitable metals described above. In more specific embodiments, the zeolite is, or includes, for example, copper-loaded ZSM5 (i.e., Cu-ZSM5), Fe-ZSM5, Cu,Fe-ZSM5, or a mixture of Cu-ZSM5 and Fe-ZSM5. In other embodiments, the zeolite is, or includes, for example, Cu—La-ZSM5, Fe—La-ZSM5, Fe—Cu—La-ZSM5, Cu—Sc-ZSM5, or Cu—In-ZSM5.

The zeolite catalyst described above is typically not coated with a metal-containing film or layer. However, the instant invention also contemplates the zeolite catalyst described above coated with a metal-containing film or layer as long as the film or layer does not substantially impede the catalyst from effectively functioning as a conversion catalyst, as intended herein. By being coated, the film or layer resides on the surface of the zeolite. In some embodiments, the surface of the zeolite refers to only the outer surface (i.e., as defined by the outer contour area of the zeolite catalyst), while in other embodiments, the surface of the zeolite refers to or includes inner surfaces of the zeolite, such as the surfaces within pores or channels of the zeolite. The metal-containing film or layer can serve, for example, to adjust the physical characteristics of the catalyst, the catalytic efficiency, or catalytic selectivity. Some examples of metal-containing surfaces include the oxides and/or sulfides of the alkali metals, alkaline earth metals, and divalent transition or main group metals, provided that such surface metals are non-contaminating to the hydrocarbon product and non-deleterious to the conversion process.

The metal (i.e., other than aluminum or silicon) in the metal-zeolite composition is nanoscopically dispersed throughout the zeolite phase. The term “nanoscopically dispersed” corresponds to the presence of agglomerations (if present) of the metal of no more than or less than 1 micron. In some embodiments, the agglomerations have a size of no more than or less than 0.9, 0.8, 0.7, 0.6, 0.5, 0.4, 0.3, 0.2, 0.1, 0.05, 0.02, or 0.01 micron. In some embodiments, the term “nanoscopically dispersed” corresponds to the metal being atomically dispersed throughout the zeolite phase, in which case no agglomeration of the metal is present. The zeolite phase may also be unagglomerated. If agglomerations of the zeolite are present, the agglomerations may independently have a size of no more than or less than 0.9, 0.8, 0.7, 0.6, 0.5, 0.4, 0.3, 0.2, 0.1, 0.05, 0.02, or 0.01 micron.

In another aspect, the present disclosure is directed to a method for producing the metal-zeolite composition. The method includes an initial step of mixing and grinding a zeolite phase, such as any of those described above, with a metal salt by a solvent-less ball milling process to produce an initial mixture. The mixing process results in the zeolite phase and metal salt being intimately mixed and ground. The metal salt is a salt of any of the metals described above, wherein the salt may be a hydrate or anhydrous. The salt may be, for example, a nitrate, sulfate, halide, hydroxide, carbonate, phosphate, carboxylate, oxide, or sulfide of any of the metals described above. Some examples of metal salts include yttrium nitrate, yttrium oxide, yttrium hydroxide, yttrium chloride, yttrium carbonate, yttrium oxalate, zirconium nitrate, niobium nitrate, copper sulfate, copper nitrate, iron oxide, iron sulfate, and iron carbonate. After the ball milling process, the initial mixture (and final metal-zeolite product after calcining) contains the metal nanoscopically dispersed throughout the zeolite phase, as described above. That is, the metal may include agglomerations of no more than 1 micron (or no more than 0.5, 0.2, 0.1, 0.05, 0.02, or 0.01 micron) in size, or the metal may not be agglomerated, instead being atomically dispersed throughout said zeolite phase. In different embodiments, agglomerations of the metal are present, but at least 80%, 85%, 90%, 95%, 98%, 99%, or 100% of the agglomerations are no more than or less than 1, 0.5, 0.2, 0.1, 0.05, 0.02, or 0.01 micron. The metal may also be partially or completely atomically dispersed, e.g., at least 80%, 85%, 90%, 95%, 98%, 99%, or 100% of the metal may be atomically dispersed.

Ball milling machines and their use for grinding materials are well known in the art. As well known, ball milling is a process in which a multiplicity of grinding balls are combined with a particulate material in a container, and the container is continuously rotated to result in continuous impacts between the balls and material, which results in a reduction in size of the material. The ball milling process reduces the size of the material to a nanoscopic size, as provided above, to result in an initial mixture (and final product) in which the metal is nanoscopically dispersed throughout the zeolite phase. Some of the conditions that may be varied in the ball milling process include the number of balls used, ball size, container (jar) size, container rotation speed (rpm), processing time, and sample quantity. As further discussed in detail below and in the Examples section, although ball milling is well known, specific conditions were herein employed to achieve the most preferably dispersed metal-zeolite compositions described herein. For example, if the grinding speed is too low, there may not be sufficient energy to disperse and mix the metal salts with the zeolites. On the contrary, if the grinding speed is too high, the metal salts may decompose during mixing due to the generation of heat, which is not beneficial for generating well-dispersed catalysts.

The ball milling process may employ a number of balls of precisely or at least, for example, 2, 3, 4, 5, 10, 15, 20, 25, 30, 35, 40, 45, 50, 55, 60, 65, 70, 75, 80, 85, 90, 95, 100, 110, 120, 130, 140, 150, 200, 250, 300, 350, 400, 450, 500, 600, 700, 800, 900, or 1000, or a number of balls bounded by any two of the foregoing values (e.g., 2-1000, 2-500, 2-200, 2-100, 2-50, 5-100, 5-50, 10-100, or 10-50).

The ball size may be precisely or at least, for example, 1, 2, 3, 4, 5, 10, 15, 20, 25, 30, 35, 40, 45, or 50 mm, or a ball size bounded by any two of the foregoing values (e.g., 1-50, 2-50, 5-50, 1-40, 2-40, 5-40, 1-30, 2-30, 5-30, 5-20, or 5-15 mm). In some embodiments, precisely or at least two different ball sizes are used, which may be independently selected from any of the sizes or ranges thereof provided above, e.g., a first ball size of 10-20 mm and a second ball size of 1-5 mm, or a first ball size of 20-50 mm and a second ball size of 2-10 mm. In the case where more than one ball size is present, the number of balls of a certain size may be independently selected from any of the number of balls provided above. For example, 2-100, 5-100, 10-100, 20-100, 30-100, 40-100, or 50-100 balls may have a first ball size while 10-1000, 20-1000, 50-1000, 100-1000, 150-1000, 200-1000, 300-1000, 400-1000, or 500-1000 balls may have a second ball size, wherein the first ball size may be larger (e.g., two-times, three-times, or four-times) than the second ball size, or the first ball size may be smaller (e.g., one-half, one-third, or one-quarter) than the second ball size. Any of the above provided ball sizes or ranges thereof may be combined with any of the above provided number of balls or ranges thereof.

The container size (i.e., volume in which the balls and material are contained) may be precisely, about, or at least, for example, 10, 20, 30, 40, 50, 60, 70, 80, 90, 100, 120, 150, 200, 250, 300, 350, 400, 450, 500, 600, 700, 800, 900, 1000, 1500, 2000, 2500, 3000, 3500, 4000, 4500, or 5000 mL, or a size within a range bounded by any two of the foregoing values (e.g., 10-5000, 10-2000, 10-1000, 10-500, 10-250, 100-5000, 100-2000, 100-1000, 100-500, or 100-250 mL). Any of the foregoing container sizes or ranges thereof may be combined with any of the ball sizes or ranges thereof and number of balls or ranges thereof provided above.

The container rotation speed (i.e., speed) may be precisely or at least, for example, 100, 120, 150, 180, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 rpm, or an rpm within a range bounded by any two of the foregoing values (e.g., 100-500, 100-450, 100-400, 100-350, 100-300, 150-500, 150-450, 150-400, 150-350, 150-300, 200-500, 200-450, 200-400, 200-350, or 200-300 rpm). Any of the foregoing speeds or ranges thereof may be combined with any of the container sizes or ranges thereof, ball sizes or ranges thereof, and number of balls or ranges thereof provided above.

The processing time is the amount of time that the container undergoes rotation during the ball milling process. The processing time may be precisely or at least, for example, 1, 2, 5, 10, 20, 30, 40, 50, 60, 70, 80, 90, 100, 110, or 120 minutes, or a processing time within a range thereof (e.g., 1-120, 5-120, 10-120, 20-120, 30-120, 1-90, 5-90, 10-90, 20-90, or 30-90 minutes). Any of the foregoing processing times or ranges thereof may be combined with any of the container rotation speeds or ranges thereof, container sizes or ranges thereof, ball sizes or ranges thereof, and number of balls or ranges thereof provided above. For example, the ball milling process may employ a speed of precisely or at least 100, 120, 150, 180, or 200 rpm for precisely or at least 5, 10, 20, or 30 minutes, or more specifically, a speed of at least 150 rpm for at least 5 minutes, or a speed of at least 200 rpm for at least 5 minutes.

The sample quantity is the amount of solid material (i.e., zeolite and metal salt) placed in the ball milling container. The sample quantity may be precisely or at least, for example, 0.5, 1, 2, 5, 10, 20, 30, 40, 50, 100, 200, 300, 400, 500, 1000, 2000, or 5000 g, or a sample quantity within a range thereof (e.g., 10-5000, 50-5000, 100-5000, 10-2000, 50-2000, 100-2000, 10-1000, 50-1000, 100-1000, 10-500, 50-500, or 100-500 g). Any of the foregoing sample quantities may be combined with any of the processing times or ranges thereof, container rotation speeds or ranges thereof, container sizes or ranges thereof, ball sizes or ranges thereof, and number of balls or ranges thereof provided above.

The method of producing the metal-zeolite composition includes a subsequent step of calcining the initial mixture to produce the final metal-zeolite composition which contains the metal nanoscopically dispersed throughout the zeolite phase. The calcination step functions to permanently incorporate the impregnated metals into the zeolite, e.g., by replacing Al⁺³ and/or Si⁺⁴ and forming metal-oxide bonds within the zeolite material. Anions originally present in the metal salt (e.g., nitrate, sulfate, or carbonate) are typically volatilized during the calcination. In different embodiments, the calcination step can be conducted at a temperature of at least 100° C., 150° C., 200° C., 250° C., 300° C., 350° C., 400° C., 450° C., 500° C., 550° C., 600° C., 650° C., 700° C., 750° C., or 800° C., or within a range therein (e.g., 400-700° C.), for a time period of, for example, 15 minutes, 30 minutes, 1 hour, 2 hours, 6 hours, 12 hours, 24 hours, 30 hours, 36 hours, or 48 hours, or within a range therein. In some particular embodiments, the calcination step is conducted at a temperature of at least 400° C., 450° C., or 500° C. for a time period of at least two, three, or four hours. In some embodiments, the calcination step includes a temperature ramping step from a lower temperature to a higher temperature. The ramp rate may be, for example, 1, 2, 5, or 10° C./min or within a range therein. For example, the calcination step may include a ramping stage from 25-500° C. or 100-500° C. at a ramp rate of 1, 2, 5, or 10° C./min. Following completion of the calcination step, the metal-zeolite composition is generally cooled to room temperature. In some embodiments, the metal-zeolite composition is cooled down gradually at a desirable rate, e.g., 1, 2, 5, or 10° C./min.

Generally, the calcination step is conducted under normal atmospheric pressure. However, in some embodiments, an elevated pressure (e.g., above 1 atm and up to 2, 5, or 10 atm) is employed, while in other embodiments, a reduced pressure (e.g., below 1, 0.5, or 0.2 atm) is employed. Furthermore, although the calcination step may be conducted under a normal air atmosphere, in some embodiments, an elevated oxygen, reduced oxygen, or inert atmosphere is used. Some gases that can be included in the processing atmosphere include, for example, oxygen, nitrogen, helium, argon, carbon dioxide, and mixtures thereof.

In another aspect, the present disclosure is directed to a method for converting an oxygen-containing organic species into a hydrocarbon fraction. The oxygen-containing organic species may be, for example, one or a combination of alcohols (e.g., methanol, ethanol, n-propanol, isopropanol, 1-butanol, 2-butanol, isobutanol, or butanediol), one or a combination of aldehydes (e.g., acetaldehyde, propionaldehyde, or butyraldehyde), one or a combination of ketones (e.g., acetone, butanone, 2-pentanone, or 3-pentanone), or a hydroxy-ketone (e.g., acetoin). In the method described, the oxygen-containing organic species is catalytically converted to a hydrocarbon fraction by contacting the oxygen-containing organic species with any of the metal-loaded zeolite catalysts described above at conditions (particularly, temperature and choice of catalyst) suitable to effect the conversion. The hydrocarbon fraction may contain a single type or a mixture of hydrocarbon molecules.

In some embodiments, the oxygen-containing organic species is or includes an alcohol. The alcohol primarily considered herein is one that can be produced by a fermentation process (i.e., a bio-alcohol). Most notable examples of bio-alcohols considered herein include ethanol, butanol, and isobutanol. In different embodiments, the alcohol can be ethanol, or butanol, or isobutanol, or a combination thereof, as commonly found in fermentation streams. In particular embodiments, the alcohol is an aqueous solution of alcohol (i.e., the alcohol is a component of an aqueous solution), as found in fermentation streams. In fermentation streams, the alcohol is typically in a concentration of no more than or less than about 20% (vol/vol), 15%, 10%, or 5%. In some embodiments, a fermentation stream is directly contacted with the catalyst (typically, after filtration to remove solids) to effect the conversion of the alcohol in the fermentation stream. In other embodiments, the fermentation stream is concentrated in alcohol (for example, of at least or up to 30%, 40%, or 50%) before contacting the fermentation stream with the catalyst. In yet other embodiments, alcohol in the fermentation stream is selectively removed from the fermentation stream, such as by distillation, to produce a substantially pure form of alcohol as the feedstock (e.g., a concentration of at least 90% or 95% of alcohol). In still other embodiments, the alcohol is completely dewatered into 100% alcohol before contacting with the catalyst.

As used herein, the term “about” generally indicates within ±0.5%, 1%, 2%, 5%, or up to ±10% of the indicated value. For example, a concentration of about 20% generally indicates in its broadest sense 20±2%, which indicates 18-22%. In addition, the term “about” can indicate either a measurement error (i.e., by limitations in the measurement method), or alternatively, a variation or average in a physical characteristic of a group.

A wide variety of hydrocarbon product can be produced by the described method. In some embodiments, the hydrocarbon fraction is or includes a saturated fraction. The saturated fraction is typically in the class of alkanes, which may be straight-chained, branched, or a mixture thereof. The alkanes particularly considered herein include those containing at least four, five, or six carbon atoms, and up to twelve, fourteen, sixteen, seventeen, eighteen, or twenty carbon atoms. Some examples of straight-chained alkanes include n-butane, n-pentane, n-hexane, n-heptane, n-octane, n-nonane, n-decane, n-undecane, n-dodecane, n-tridecane, n-tetradecane, n-pentadecane, n-hexadecane, n-heptadecane, n-octadecane, n-nonadecane, and n-eicosane. Some examples of branched alkanes include isobutane, isopentane, neopentane, isohexane, 3-methylpentane, 2,3-dimethylbutane, 2,2-dimethylbutane, 2-methylhexane, 3-methylhexane, 2,2-dimethylpentane, 2,3-dimethylpentane, 2,4-dimethylpentane, 3,3-dimethylpentane, 2-methylheptane, and 2,2,4-trimethylpentane (isooctane). In other embodiments, the hydrocarbon fraction is or includes an unsaturated fraction. The unsaturated fraction may include an aliphatic or aromatic fraction. The unsaturated fraction may include olefins (i.e., alkenes, such as, for example, ethylene, propylene, n-butene, and/or isobutene) and aromatics (for example, naphthalene, benzene, toluene, and/or xylenes). In some embodiments, any one or more classes or specific types of hydrocarbon product described above may be substantially or completely excluded as a product.

In particular embodiments, the hydrocarbon product is a mixture of hydrocarbon compounds useful as a fuel or as a blendstock in fuel. The mixture of hydrocarbon compounds preferably substantially corresponds (e.g., in composition and/or properties) to a known petrochemical fuel, such as petroleum, or a fractional distillate of petroleum. Some examples of petrochemical fuels include gasoline, kerosene, diesel, and jet propellant (e.g., JP-8). Like hydrocarbon fuel grades in current use, the mixture of hydrocarbon compounds produced herein can, in some embodiments, be predominantly or exclusively composed of alkanes, alkenes, aromatics, or a mixture thereof. Although aromatics (particularly benzene) may be present in the hydrocarbon mixture, their presence may be minimized (e.g., no more than or less than 1, 0.5, 0.3, 0.2, or 0.1 wt %) to adhere to current fuel standards. The raw hydrocarbon product, produced by the instantly described method, is typically fractionated by distillation into different fuel grades, each of which is known to be within a certain boiling point range. A particular advantage of the instant method is its ability to produce such fuel grades in the substantial absence of contaminants (e.g., mercaptans) normally required to be removed during the petroleum refining process. Moreover, by appropriate adjustment of the catalyst and processing conditions, a select distribution of hydrocarbons can be obtained.

Depending on the final composition of the hydrocarbon product, the product can be directed to a variety of other applications, including, for example, as precursors for plastics, polymers, and fine chemicals. The process described herein can advantageously produce a range of hydrocarbon products that differ in any of a variety of characteristics, such as molecular weight (i.e., hydrocarbon weight distribution), degree of saturation or unsaturation (e.g., alkane to alkene ratio), and level of branched or cyclic isomers. The process provides this level of versatility by appropriate selection of, for example, composition of the catalyst (e.g., catalytic metal), amount of catalyst (e.g., ratio of catalyst to alcohol precursor), processing temperature, and flow rate (e.g., LHSV).

In the process, a suitable reaction temperature is employed during contact of the oxygen-containing organic species with the catalyst. Generally, the reaction temperature is at least 100° C. and up to 550° C. In different embodiments, the reaction is precisely or about, for example, 100° C., 125° C., 150° C., 175° C., 200° C., 225° C., 250° C., 275° C., 300° C., 325° C., 350° C., 375° C., 400° C., 425° C., 450° C., 475° C., 500° C., 525° C., or 550° C., or a temperature within a range bounded by any two of the foregoing exemplary temperatures, e.g., 100° C.-550° C., 200° C.-550° C., 300° C.-550° C., 400° C.-550° C., 450° C.-550° C., 100° C.-500° C., 200° C.-500° C., 300° C.-500° C., 350° C.-500° C., 400° C.-500° C., 450° C.-500° C., 100° C.-450° C., 200° C.-450° C., 300° C.-450° C., 350° C.-450° C., 400° C.-450° C., 100° C.-425° C., 200° C.-425° C., 300° C.-425° C., 350° C.-425° C., 375° C.-425° C., 400° C.-425° C., 100° C.-400° C., 200° C.-400° C., 300° C.-400° C., 350° C.-400° C., and 375° C.-400° C.

Generally, ambient (i.e., normal atmospheric) pressure of about 1 atm is used in the method described herein. However, in some embodiments, an elevated pressure or reduced pressure may be used. For example, in some embodiments, the pressure may be elevated to, for example, 1.5, 2, 3, 4, or 5 atm, or reduced to, for example, 0.5, 0.2, or 0.1 atm.

The catalyst and reactor can have any of the designs known in the art for catalytically treating a fluid or gas at elevated temperatures, such as a fluidized bed reactor. The process may be in a continuous or batch mode. In particular embodiments, the oxygen-containing organic species is injected into a heated reactor such that the oxygen-containing organic species is quickly volatilized into gas, and the gas passed over the catalyst. In some embodiments, the reactor design includes a boiler unit and a reactor unit if the fermentation stream is used directly as a feedstock without purification. The boiler unit is generally not needed if the fermentation stream is distilled to concentrate the oxygen-containing organic species because the distillation process removes the dissolved solids in the fermentation streams. The boiler unit volatilizes liquid feedstock into gases prior to entry into the reactor unit and withholds dissolved solids.

In some embodiments, the conversion method described above is integrated with a fermentation process, wherein the fermentation process produces the oxygen-containing organic species used as feedstock for the conversion process. By being “integrated” is meant that oxygen-containing organic species produced at a fermentation facility or zone is sent to and processed at a conversion facility or zone (which performs the conversion process described above). Preferably, in order to minimize production costs, the fermentation process is in close enough proximity to the conversion facility or zone, or includes appropriate conduits for transferring produced oxygen-containing organic species to the conversion facility or zone, thereby not requiring the oxygen-containing organic species to be shipped. In particular embodiments, the fermentation stream produced in the fermentation facility is directly transferred to the conversion facility, generally with removal of solids from the raw stream (generally by filtration or settling) before contact of the stream with the catalyst.

In some embodiments, the fermentation process is performed in an autonomous fermentation facility, i.e., where saccharides, produced elsewhere, are loaded into the fermentation facility to produce alcohol. In other embodiments, the fermentation process is part of a larger biomass reactor facility, i.e., where biomass is decomposed into fermentable saccharides, which are then processed in a fermentation zone. Biomass reactors and fermentation facilities are well known in the art. Biomass often refers to lignocellulosic matter (i.e., plant material), such as wood, grass, leaves, paper, corn husks, sugar cane, bagasse, and nut hulls. Generally, biomass-to-ethanol conversion is performed by 1) pretreating biomass under well-known conditions to loosen lignin and hemicellulosic material from cellulosic material, 2) breaking down cellulosic material into fermentable saccharide material by the action of a cellulase enzyme, and 3) fermentation of the saccharide material, typically by the action of a fermenting organism, such as suitable yeast.

In other embodiments, an alcohol is produced from a more direct sugar source, such as a plant-based source of sugars, such as sugar cane or a grain starch (such as corn starch). Ethanol production via corn starch (i.e., corn starch ethanol) and via sugar cane (i.e., cane sugar ethanol) currently represent some of the largest commercial production methods of ethanol. Integration of the instant conversion process with any of these large scale ethanol production methods is contemplated herein.

Examples have been set forth below for the purpose of illustration and to describe certain specific embodiments of the invention. However, the scope of this invention is not to be in any way limited by the examples set forth herein.

### EXAMPLES

Overview

The methods disclosed herein provide an environmentally friendly approach of mechanochemistry for controlled synthesis of metal-zeolite materials. The methods feature simplicity, scalability, high-yield, high-producibility, and cost-effectiveness.

The experiments include: 1) synthesis of metal-doped zeolite catalyst via ball milling, and 2) ethanol upgrading or carbon-carbon coupling reaction over the synthesized metal-zeolite catalyst.

Preparation of Metal-Zeolite Composition

Mechanochemical preparation of metal-zeolites using a ball mill machine included the following steps:

a) Mixed the zeolite and metal precursors in an appropriately sized grinding jar;

b) Ground the mixture with controlled speed and time;

c) Calcined the resulting solids in the tubular furnace under air flow; and

d) Cooled the solid material to room temperature followed by pelletization and grinding in preparation for catalytic reactions.

Several conditions (e.g., sample quantity, grinding jar size, number of balls, size of the balls, speed (rpm), and time) were varied, as shown in the following table:

TABLE 1
Controlled conditions for synthesizing highly dispersed
metals embedded in zeolite via mechanochemistry

| | | Number of Balls | | | |
|---|---|---|---|---|---|
| Sample | Grinding Jar | Ball size | Ball size | Speed | Time |
| quantity (g) | size (mL) | (15 mm) | (5 mm) | (rpm) | (mins) |
| 0.5-5 | 12-50 | 1-6 | 10-50 | 230-300 | 10-30 |
| 5-10 | 50-125 | 5-20 | 30-150 | 250-350 | 15-30 |
| 10-20 | 80-250 | 10-50 | 100-250 | 300-400 | 20-40 |
| 20-80 | 125-500 | 40-100 | 200-1000 | 300-400 | 30-90 |

The incorporated metals can be any one or more metals that provide catalytic activity or other useful characteristic. For example, in some embodiments, the metals can be selected from the lanthanide metals, and Cu, Zn, Fe, Al in the form of salts (e.g., metal nitrates). Dealuminated H type zeolites (such as H-Beta, and H-ZSM-5) may be used as the zeolite phase, preferably with a Si/Al ratio greater than 1000.

Example 1. In particular experiments, 0.1296 gram yttrium nitrate hexahydrate (97%) was mixed with 1 gram DeAl-Beta (Si/Al≅1300) in a 50 mL zirconium oxide coated stainless steel jar. Thirty (30) grinding balls with two different sizes were placed in the grinding jar. The mixture was milled at 230 rpm for 20 minutes at room temperature. The collected solid was calcined at 550° C. (1° C./min) for 6 hours in the tube oven under 10 cm³ g cat⁻¹s⁻¹ air flow (99.99%), and then cooled down to room temperature. The resulting sample is assigned to 3 wt % Y-Beta.

Example 2. 0.1516 or 0.4041 gram ytterbium(III) nitrate pentahydrate was mixed with 1 gram DeAl-Beta ((Si/Al≅1300) in a 50 mL zirconium oxide coated stainless steel jar. Thirty-five (35) grinding balls with two different sizes were placed in the grinding jar. The mixture was milled at 250 rpm for 15 minutes at room temperature. The collected solid was calcined at 550° C. (1° C./min) for 6 hours in the tube oven under 10 cm³ g cat⁻¹s⁻¹ air flow (99.99%), and then cooled down to room temperature. The resulting samples are designated as 5.8 wt % La-Beta and 15.6 wt % Yb-Beta, respectively.

Example 3. 0.046 gram nickel (II) nitrate hexahydrate was mixed with 1 gram DeAl-Beta ((Si/Al≅1300) in a 50 mL zirconium oxide coated stainless steel jar. Thirty (30) grinding balls with two different sizes were placed in the grinding jar. The mixture was milled at 230 rpm for 20 minutes at room temperature. The collected solid was calcined at 550° C. (1° C./min) for 6 hours in the tube oven under 10 cm³ g cat⁻¹s⁻¹ air flow (99.99%), and then cooled down to room temperature. The resulting sample is designated as 1 wt % Ni-Beta.

Example 4. 0.0760-gram copper (II) nitrate trihydrate was mixed with 2 gram DeAl-Beta ((Si/Al≅1300) in a 50 mL zirconium oxide coated stainless steel jar. Forty (40) grinding balls with two different sizes were placed in the grinding jar. The mixture was milled at 250 rpm for 20 minutes at room temperature. The collected solid was calcined at 550° C. (1° C./min) for 6 hours in the tube oven under 10 cm³ g cat⁻¹s⁻¹ air flow (99.99%), and then cooled down to room temperature. The resulting sample is designated as 1 wt % Cu-Beta.

Example 5. 0.0380-gram copper (II) nitrate trihydrate, 0.0902 gram zinc nitrate hexahydrate, and 0.2498 gram lanthanum nitrate hexahydrate were mixed with 1 gram DeAl-Beta ((Si/Al≅1300) in a 50 mL zirconium oxide coated stainless steel jar. Thirty (40) grinding balls with two different sizes were placed in the grinding jar. The mixture was milled at 230 rpm for 15, and followed by 260 rpm for 5 minutes at room temperature. The collected solid was calcined at 550° C. (1° C./min) for 6 hours in the tube oven under 10 cm³ g cat⁻¹s⁻¹ air flow (99.99%), and then cooled down to room temperature. The resulting sample is designated as 1 wt % Cu-2 wt % Zn-7 wt % La-Beta.

Example 6. 0.114-gram copper (II) nitrate trihydrate and 1.034 yttrium nitrate hexahydrate were mixed with 3 gram DeAl-Beta ((Si/Al≅1300) in a 50 mL zirconium oxide coated stainless steel jar. Thirty (50) grinding balls with two different sizes were placed in the grinding jar. The mixture was milled at 280 rpm for 20 mins, and followed by 300 rpm for 5 minutes at room temperature. The collected solid was calcined at 550° C. (1° C./min) for 6 hours in the tube oven under 10 cm³ g cat⁻¹s⁻¹ air flow (99.99%), and then cooled down to room temperature. The resulting sample is designated as 1 wt %-7 wt % Y-Beta.

Example 7. 0.0380 gram copper (II) nitrate trihydrate was mixed with 1 gram NbO(PO₄) in a 50 mL zirconium oxide coated stainless steel jar. Thirty (30) grinding balls with two different sizes were placed in the grinding jar. The mixture was milled at 230 rpm for 15 minutes at room temperature. The collected solid was calcined at 550° C. (1° C./min) for 6 hours in the tube oven under 10 cm³ g cat⁻¹s⁻¹ air flow (99.99%), and then cooled down to room temperature. The resulting sample is designated as 1 wt % Cu—NbO(PO₄).

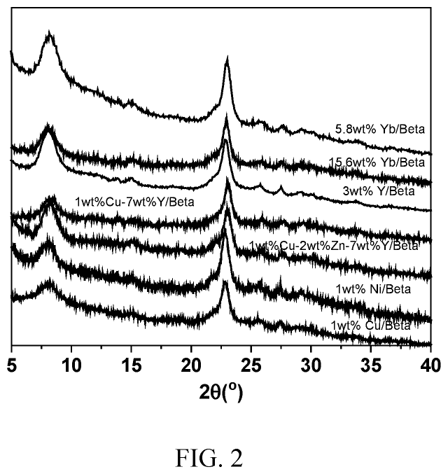

The as-synthesized mono-metallic, bimetallic, and trimetallic catalysts were characterized by x-ray diffraction (XRD). XRD patterns (FIG. 2) indicate that the primary Beta structure was well preserved after introduction of metals via ball-milling method. The lack of metal oxide peaks for all samples demonstrate that all the metal particles were either well dispersed or present as non-crystalline species into the zeolite supports.

Conversion of Oxygen-Containing Organic Species to Hydrocarbons

The above synthesized metal-doped zeolite was particularly applied for biomass-derived ethanol conversion to olefins, which are important precursors to sustainable aviation fuels. Ethanol conversion was conducted in a tubular quartz reactor (12.7 mm O.D.) with a fixed bed configuration under ambient pressure. The reactor was vertically aligned in a temperature-controlled tubular furnace. The catalyst bed temperature was measured with a K-type thermocouple. Catalysts were pelletized and ground into defined particle sizes. The preferred reaction temperature was from 250 to 400° C.

A benchmarking reaction (ethanol upgrading to olefins) was performed over 1 wt % Cu-7 wt % Y/Beta catalysts synthesized via hand grinding at 1 gram scale (1Cu-7Y/Beta-GH-1) and 2 gram scale (1Cu-7Y/Beta-GH-2), as well as via ball milling at 3 gram scale (1Cu-7Y/Beta-BM-3). Typically, 0.3 g of each catalyst was pretreated by heating in He flow at 400° C., followed by reduction in H₂ at 350° C. before measurements of catalytic performance. Pure ethanol (C₂H₅OH, >99%) was fed with a syringe pump and vaporized inside the 3.175 mm o.d. stainless steel transfer lines heated to 150° C. using electrical heating tapes. The products were analyzed with an online gas chromatograph equipped with both a flame ionization detector and thermal conductivity detectors.

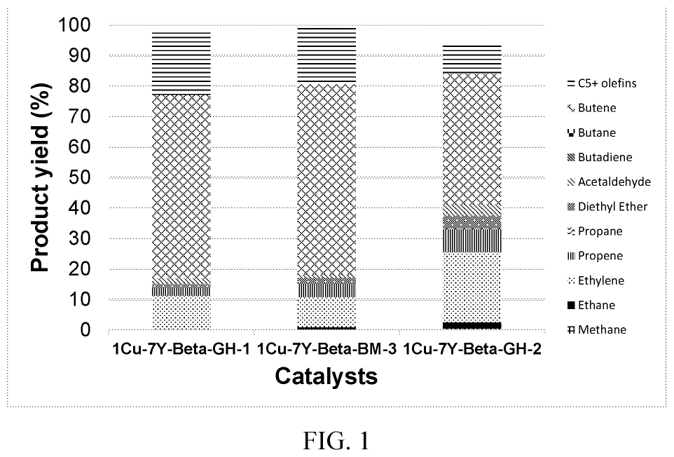

The benchmark experimental results from the ethanol upgrading are shown in FIG. 1. The results demonstrate that the catalytic performance over the mechanochemically synthesized 1Cu-7Y/Beta-BM-3 at 3 gram scale is slightly better than the conventional hand-grinding prepared 1Cu-7Y/Beta-GH-1 (GH for hand-grinding) at 1 gram scale in terms of the yield of C₃⁺ olefins. Notably, the mechanochemically synthesized 1Cu-7Y/Beta-BM-3 provides a substantially better yield of C₃⁺ olefins compared to 1Cu-7Y/Beta-GH-2 prepared by hand-grinding at 2 gram scale. The 1Cu-7Y/Beta-GH-2 catalyst prepared by hand-grinding generates significantly higher byproduct ethylene. As shown by the above evidence, the mechanochemical process provides particularly significant advantages in larger scale syntheses of catalysts. The synthesis method described herein not only provides superior mixing and integration of components, but also generates sufficient heat to better disperse the metal precursors during the synthesis. The proper control of jar size, ball size, ball numbers, milling speed, and time were carefully selected to achieve the most desirably homogeneous and well-dispersed metal-zeolite compositions.

Significantly, operational parameters that generate too much heat may decompose the metal precursors before mixing and dispersing the metal precursors, while those that generate less heat may not be able to sufficiently disperse the metal precursors. Thus, parameters of the ball milling process were judiciously selected to provide a proper balance of heat to result in superior dispersal of the metal in the zeolite phase. The mechanochemical process described herein can be widely applied in the synthesis of monometallic, bimetallic, or multi-metallic doped zeolite catalytic materials, any of which may be used in biomass-derived alcohols conversion. The process described herein possesses several additional advantages including scalability, high-yield, high-producibility, and cost-effectiveness.

While there have been shown and described what are at present considered the preferred embodiments of the invention, those skilled in the art may make various changes and modifications which remain within the scope of the invention defined by the appended claims.

What is claimed is:

1. A metal-zeolite composition comprising:
(i) a zeolite phase; and
(ii) a metal, other than aluminum or silicon, nanoscopically dispersed throughout said zeolite phase, wherein, if agglomerations of said metal are present, the agglomerations have a size of no more than 1 micron.

2. The composition of claim 1, wherein said zeolite phase is a dealuminated zeolite phase.

3. The composition of claim 1, wherein said zeolite phase is a ZSM-5 zeolite phase.

4. The composition of claim 1, wherein said metal is selected from transition metals.

5. The composition of claim 1, wherein said metal is selected from main group metals.

6. The composition of claim 1, wherein said metal is selected from lanthanide metals.

7. The composition of claim 1, wherein, if agglomerations of said metal are present, the agglomerations have a size of no more than 0.5 micron.

8. The composition of claim 1, wherein, if agglomerations of said metal are present, the agglomerations have a size of no more than 0.1 micron.

9. The composition of claim 1, wherein agglomerations of said metal are not present, and said metal is atomically dispersed throughout said zeolite phase.

10. The composition of claim 1, wherein the metal-zeolite composition is produced by a process in which said zeolite phase and a metal salt containing said metal are intimately mixed and ground in a solvent-less ball milling process followed by calcination.

11. The composition of claim 10, wherein said ball milling process employs a speed of at least 150 rpm for at least 5 minutes.

12. The composition of claim 10, wherein said ball milling process employs a speed of at least 200 rpm for at least 5 minutes.

13. The composition of claim 10, wherein said calcination is conducted at a temperature of at least 400° C. for at least 3 hours.

14. A method for producing a metal-zeolite composition, the method comprising:
(i) mixing and grinding a zeolite phase and a metal salt by a solvent-less ball milling process to produce an initial mixture; and
(ii) calcining said initial mixture to produce said metal-zeolite composition, wherein the metal in said metal-zeolite composition is nanoscopically dispersed throughout said zeolite phase, wherein, if agglomerations of said metal are present, the agglomerations have a size of no more than 1 micron.

15. The method of claim 14, where said zeolite phase is a dealuminated zeolite phase.

16. The method of claim 14, wherein said zeolite phase is a ZSM-5 zeolite phase.

17. The method of claim 14, wherein said metal is selected from transition metals.

18. The method of claim 14, wherein said metal is selected from main group metals.

19. The method of claim 14, wherein said metal is selected from lanthanide metals.

20. The method of claim 14, wherein, if agglomerations of said metal are present, the agglomerations have a size of no more than 0.5 micron.

21. The method of claim 14, wherein, if agglomerations of said metal are present, the agglomerations have a size of no more than 0.1 micron.

22. The method of claim 14, wherein agglomerations of said metal are not present, and said metal is atomically dispersed throughout said zeolite phase.

23. The method of claim 14, wherein said solvent-less ball milling process employs a speed of at least 150 rpm for at least 5 minutes.

24. A method for converting an oxygen-containing organic species into a hydrocarbon fraction, the method comprising contacting the oxygen-containing organic species with a metal-containing zeolite catalyst at a temperature of at least 100° C. and up to 550° C. to result in said oxygen-containing organic species being converted to said hydrocarbon fraction, wherein said metal-containing zeolite catalyst comprises the following components:
(i) a zeolite phase; and
(ii) a metal, other than aluminum or silicon, nanoscopically dispersed throughout said zeolite phase, wherein, if agglomerations of said metal are present, the agglomerations have a size of no more than 1 micron.

25. The method of claim 24, wherein said zeolite phase is dealuminated.

26. The method of claim 24, wherein the zeolite phase is a ZSM-5 zeolite phase.

27. The method of claim 24, wherein the metal is selected from the group consisting of copper, zinc, iron, lanthanide metals, and combinations thereof.
